(19) United States
(12) Patent Application Publication
(10) Pub. No.: US 2004/0009220 A1
(43) Pub. Date: Jan. 15, 2004

(54) Rate-controlled beta blockers and process for forming same

(76) Inventor: Vinay Sharma, Long Valley, NJ (US)

Correspondence Address: Louis E. Marn, 9 Edgewater Drive, Palm Coast, FL, 32164, US

(21) Appl. No.: 10/334,194

(22) Filed: Dec. 31, 2002

Related U.S. Application Data
(60) Provisional application No. 60/345,030, filed on Dec. 31, 2001.

(51) Int. Cl. A61K 31/724; A61K 9/26

(52) U.S. Cl. 424/470; 514/58

(57) ABSTRACT

There is disclose a rate controlled delivery system for a beta-blocker form by extruding a blend of the beta-blocker, a filler, a binder/defoamer and a blend of polymers which have been intimately mixed in a densification and extrusion molding to provide a quasi-monolithic compact mass which erodes over a period of from 12 to 16 hours thereby to provide therapeutic performance for a period of 24 hours.

### RELATED APPLICATION

This application claims the benefit of U.S. Provisional application No. 60/345,030 filed Dec. 31, 2001.

### BACKGROUND OF THE INVENTION

1. Field of the Invention

The present invention relates to a drug delivery system, and more particularly to a process for forming rate-controlled beta blockers and the product obtained thereby.

2. Description of the Prior Art

Metroprolol has a molecular weight of 267.37, log(P) is 1.65, pKa is 9.7 for the secondary amine. The molecular weight of the metoprolol succinate 652.8 which is freely soluble in water. In man, the absorption is rapid and complete. The plasma half life ranges from three to seven hours. The absorption profile is reported as 0% in stomach, 33% in the duodenum, 37% in the jejunum and 25% in the ileum and about 3% in the colon.

Toprol-XL is a beta, selective (cardioselective) adrenoceptor blocking agent for oral administration, available as extended release tablets. The tablets comprise a multiple unit system containing metoprolol succinate in a multitude of controlled release pallets. The tablets contain 47.5 mg, 95 mg and 190 mg of metoprolol succinate equivalent to 50, 100 and 200 mg of metoprolol tartarate, USP, respectively. Gastrointestinal transit of a multiple unit formulation (metoprolol CR/ZOK) and a non disintegrating tablet with the emphasis on the colon Abrahamsson, B. et. al, Int. J Pharm: 1996: 140(Aug 30);228-235). Beads which are coated with a rate controlling membrane and then compacted into a single nit delivery system could conceivably provide either an intact tablet or a multiparticulate system during transit. The compaction operation however could cause damage to the controlling membrane or to the bead cores. Additionally, the distribution of the beads during the blending of the beads with the excipients lends difficulty in terms of product uniformity and quality control. The advantage of compressed multiparticulates is perceived in providing a uniform movement, prandial and motility independent performance. However, the design is complicated because the basis problem is that without sufficient plasticity of the film, the coating could be destroyed under pressure and the rate control will be lost.

Atenolol, is a beta selective (cardioselective) adrenoreceptor blocking agent with a molecular weight of 266. It is a relatively polar hydrophilic compound with a water solubility of 26.5 ng/ml at 37° C. and a log partition coefficient (octanol/water) of 0.23. It is freely soluble in 1 N HCl (300 mg/ml at 25° C.). The pKa of atenolol is 9.6. The absorption profile is 12% in the duodenum, 19%%in the jejunum and 38% combined in the ileum/colon, the cumulative portal absorption is 69%.

### OBJECTS OF THE PRESENT INVENTION

An object of the present invention is to provide an improved drug delivery system for beta-blockers.

Another object of the present invention is to provide an effective once a day delivery system for a beta-blocker.

Still another object of the present invention is to provide an improved process for preparing a once daily delivery system for a beta-blocker.

### SUMMARY OF THE INVENTION

These and other objects of the present invention are achieved by a release rate of the beta-blocker is controlled based on simulation and modeling of plasma concentration versus time and gastrointestinal absorption profile to attain bioequivalency of the innovated drug Beloc-Zok®.

### BRIEF DESCRIPTION OF THE DRAWINGS

A better understanding of the present invention will be obtained by consideration of the following detailed description thereof when taken with the accompanying drawings wherein:

FIG. 1. is an UIR from IV data, Fasted State, Dog Model CRS 19999/6362; and

FIG. 2. is a synoptic plot of geometric mean concentration of metoprolol vs. time (hr) 95 mg Beloc Zok®, 100 mg extrudate; CRS, 1999,6348; and

FIG. 3. is an in-vivo rates and cumulative amounts of metoprolol (CRS 1999 6348); and

FIG. 4. is cumulative amount of metoprolol fitted to a six degree polynomial and rates obtained form the fitted cumulative; and

FIG. 5. is a monophasic design of metoprolol succinate Based on fitted in-vivo cumulative trend of Beloc Zok®.

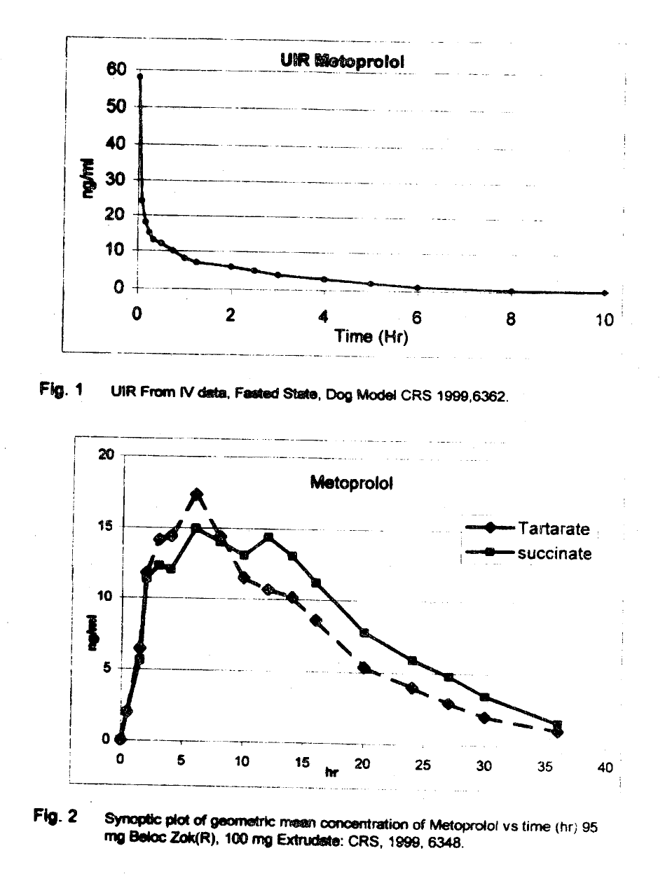

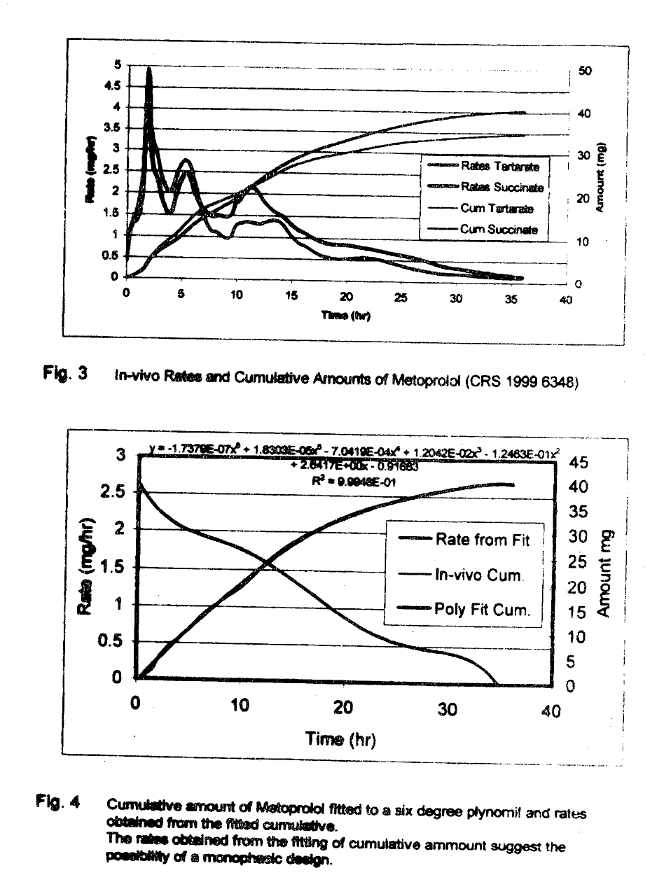

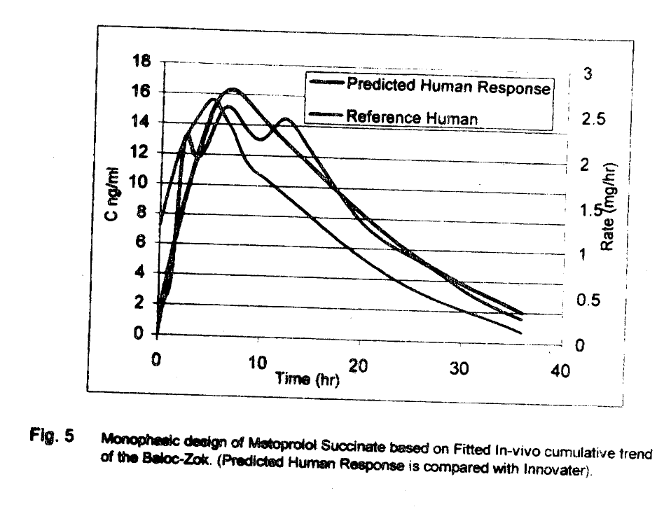

### DETAILED DESCRIPTION OF THE INVENTION

The monophasic design is based on curve fitting of the cumulative in-vivo amounts obtained by deconvolution of the human response of the innovator. The monophasic design is achieved by blending the active ingredient with the filler beta-cyclodextrin and release controlling polymers massed with hydroxypropyl cellulose dispersion in water serving as a binder and appropriately defoamed with simethicone using a planetary mixer. The damp mass of the active, filler, binder, polymer and defoamer is extruded with the extrudates dried, sized, lubricated and compressed using appropriate tooling. The resulting intimate mixing of ingredients, densification, and extrusion-molding of polymers provides a quasi monolithic compact mass which erodes slowly.

The fundamental objective of in vitro/in vivo (IVIVC) correlation is to relate in vitro drug release with the drug release in vivo. Deconvolution is a mathematical tool by which in vivo drug release may be estimated. Convolution permits prediction of an expected human response given an in-vivo release rate. The combination of IVIVC and convolution permits the prediction of an in-vivo response given an in vitro release rate. Similarly, given a human response using IVIVC and the in-vivo release rates, the in vitro release rates can be estimated.

A first step an fractional release time (FRT) approach is to establish an IVIVC is to obtain in viv0 release through the deconvolution of a plasma-time profile of the original product. The next step is to convert the rates thus obtained into cumulative amounts. Similar cumulative information is generated on in vitro rates of the original product using suitable dissolution conditions. From the corresponding cumulative amounts of in vitro and in vivo dated, fractional release times, respectively are computed. A Cartesian coordinate plot is prepared by plotting FRT (vivo) on the X-axis and FRT (in vitro) on the Y-axis which provides the IVIVC for the original product. For a simple relationship, the function can be approximated to be a straight line, Y=m×+c. Under certain conditions, the relationship may not be that simple and functions like a polynomial, polyexponential, etc. can be used.

For designing a bioequivalent product, a similar procedure is carried out for the test product with in turn yields an IVIVC for the test product. Such an approach allows an estimate of the target FRT vitro for the test based on the expected FRT vivo of the original product.

In the absence of acceptable dissolution conditions to match the time spans of in vitro/in vivo release data, scaling of the time is needed which can be achieved by compression of either the in vitro or in vivo time scales. Appropriate transformation function may be needed if the in vitro drug release deviates from the in vivo release due to physiological conditions.

The present invention is a quasi-monolithic monophasic system which is comprised of metoprolol succinate appropriated mixed with the excipients and an extrudable polymer. The drug deliver system erodes slowly over a period of from 12-16 hours.

More specifically, metoprolol succinate is incorporated into a matrix comprising a hydrophilic polymer, such as hydroxy ethyl cellulose, hydroxy propyl cellulose, hydroxy propyl methyl cellulose and polyoxyethylene homopolymers and unmicronized β-cyclodextrin. A blend is prepared in a planetary mixer by adding the binder dispersion to the powder blend. The blend is processed through an extruder consisting of a twin screw system which discharges a wet plastic material through axially positioned screens. Actual extrusion is performed by an extruding roll which forces the damp material through the screens. The feed screws and extruding rolls are chain driven through a variable speed drive (20-85 RPM). The extruded material is dried on a tray in a hot oven or a vacuum oven. The dried material is milled using an appropriate milling equipment, blended with a lubricant in a dry blender with intermeshing motion for a predetermined time, and compressed into a core using an appropriately sized tooling equipment on rotary compression machine.

Hydrophilic polymers suitable for extrusion-granulation include cellulosic derivative, such as hydroxypropylmethyl cellulose (Methocel® F and F being preferred).

### EXAMPLES OF THE PRESENT INVENTION

The following are examples of the present invention:

### Example I

EXAMPLE I

| INGREDIENTS | AMOUNT MG/core |
|---|---|
| Metroprolol succinate | 190 |
| Beta-cyclodextrin | 152 |
| Hydroxypropylmethyl Cell | 121.6 |
| (10000 Cps) | |
| Hydroxypropylmethyl Cell (50 cps) | 30.4 |
| Binding/Antifoam-hydroxypropyl | |
| Cellulose (6% w/w dispersion) | 14.82 |
| Lubricants-magnesium stearate | |
| Stearic acid | 8.63 |
| Total Core Weight | 516.45 |

### Example II

EXAMPLE II

| INGREDIENTS | AMOUNT MG/core |
|---|---|
| Atenolol | 100 |
| Beta-cyclodextrin | 80 |
| Hydroxyethyl cell high vis. | 64 |
| Hydroxyethyl cell low vis. | 16 |
| Binding Agent-hydroxypropyl | |
| Cellulose (6% w/w/dispersion) | 7.8 |
| Lubricants-mag stea/St Acid | 4.02 |
| | 271.82 |

While the present invention has been described in connection with exemplary embodiments thereof, it will be understood to those of ordinary skills that many modifications are apparent; and that this application is intended to cover any adaptations or variations thereof. Therefore, it is manifestly intended that this invention be only limited by the claims and the equivalents thereof.

What is claimed:

1. A rate controlled delivery system for a beta-blocker including a filler and a release controlling polymer prepared by a granulation process yielding a quasi-monolithic compact mass which is slowly dissolved by erosion over a period of from 12 to 16 hours is simulated gastric fluid and simulated intestinal fluid.

2. The rate controlled delivery system as defined in claim 1 wherein said beta-blocker is selected from the group consisting of metoprolol succinate or atenolol.

3. The rate controlled delivery system as defined in claim 3 wherein said beta-blocker is metoprolol succinate.

4. The rate controlled delivery system as defined in claim 1 wherein said filler is selected from the group consisting of β-cyclodextrin, methyl-β-cyclodextrin, hydroxypropyl-β-cyclodextrin, hydroxyethyl-βcyclodextrin or sulfobutylether-β-cyclodextrin.

5. The rate controlled delivery system as defined in claim 1 where in said release controlling blends of polymers are selected from the group consisting of high viscosity hydroxypropylmethyl cellulose and low viscosity hydroxypropylmethyl cellulose.

6. The rate controlled delivery system as defined in claim 1 wherein the drug/filler/polymer blend ratio is from 1:1:1.

7. The rate controlled delivery system as defined in claim 6 wherein a filler/polymer blend ratio is from 1:3 to 3:1.

8. The rate controlled delivery system as defined in claim 5 wherein a ratio of said high viscosity polymer to low viscosity polymer is 4:1 to 1:1.

9. A process for preparing a rate controlled form of a beta-blocker with comprises forming a damp mass of said beta-blocker, a filler and a release controlling polymers by granulating with an aqueous dispersion of a low viscosity hydroxypropylmethyl cellulose in a planetary mixer and extruding the resulting damp mass to form pharmaceutical dosage units.
